(19) United States
(12) Patent Application Publication
(10) Pub. No.: US 2003/0023208 A1
(43) Pub. Date: Jan. 30, 2003

(54) Apparatus for vascular access

(76) Inventors: Thomas P. Osypka, Palm Harbor, FL (US); Ronald van den Nieuwenhof, Odessa, FL (US)

Correspondence Address: CUMMINGS AND LOCKWOOD, GRANITE SQUARE, 700 STATE STREET, P O BOX 1960, NEW HAVEN, CT, 06509-1960, US

(21) Appl. No.: 09/911,563

(22) Filed: Jul. 24, 2001

(51) Int. Cl. A61M 5/00

(52) U.S. Cl. 604/256; 606/108

(57) ABSTRACT

An apparatus for vascular access is disclosed which includes a vascular access port defining an elongated tubular body of predetermined length with a central lumen having opposed proximal and distal end portions, the distal end portion adapted and configured for introduction into a blood vessel, the apparatus further including an elongated cylindrical plug body dimensioned and configured for insertion into the central lumen of the vascular access port, the plug body having a length that is substantially equal to the length of the vascular access port so as to prevent blood flow into the lumen of the access port when the plug is engaged therein.

### BACKGROUND OF THE INVENTION

1. Field of the Invention

The subject invention relates to an apparatus for vascular access, and more particularly, to an apparatus for facilitating vascular access during hemodialysis procedures.

2. Background of the Related Art

Various medical procedures, including hemodialysis, involve the surgical installation of one or more ports or catheters to facilitate access to the vasculature and hence, the blood flow of a patient. For example, in hemodialysis, blood is removed from the body, pumped through a dialysis machine so as to remove toxins from the blood, and then returned to the patient.

Vascular access for hemodialysis can be achieved in one of several ways. One prior art method for obtaining vascular access for hemodialysis is accomplished by implanting two percutaneous ports in the arm of the patient. The distal ends of the ports are disposed within the patient's veins and the proximal ends of the ports are connected directly to and in series with the dialysis equipment. One port serves as an outlet port, providing a conduit for removing blood from the patient's vasculature. The other port serves as an inlet or a conduit for returning purified blood to the patient. Hemodialysis patients must have toxins removed from their blood several times per week, and as a result, the access ports remain implanted. During periods in which dialysis is suspended, the ports are capped to prevent leakage and infection.

U.S. Pat. No. 4,417,890 to Dennehey et al. discloses a method of capping a dialysis conduit or catheter. The capped fluidic connector disclosed therein is positioned on the end of a flexible conduit, with liquid antiseptic such as povidone iodine situated within the closure, bathing the connector in antiseptic and thus sterilizing it during storage. A disadvantage to this cap, as well as other prior art closure systems, is that it is not inserted into the lumen of the access port, extending over its entire length. Consequently, stagnant blood may remain in the lumen of the vascular access port. Over time, this stagnation causes residual debris to accumulate in the lumen of the access port, reducing the dialysis flow rate and causing clotting. When the flow rate is reduced to an inefficient level, the access port must be removed and a new access port must be surgically installed in another location. This process causes the patient a great deal of discomfort. More importantly, after repeated relocations, vein thrombosis may result, making routine blood draws difficult. Eventually, a suitable site for access to blood flow can not be found and hemodialysis becomes impossible.

Another prior art method for hemodialysis involves forming an arteriovenous fistula between an artery and a vein, usually in the arm of the patient. The forming of the fistula causes the vein to enlarge, thus making it ideal for the insertion of a catheter. With this method, the vein must be repeatedly pierced. The repeated piercing of the vein causes scar tissue to develop and eventually leads to clotting of the fistula. When this occurs, surgery to insert a new fistula replacing the old fistula must again be performed. As the kidneys of a patient are unable to remove toxins, the veins are caused to become inflamed and thus it becomes difficult to find a suitable vein for the insertion of the fistula.

Therefore, it would be beneficial to provide a vascular access system that overcomes the deficiencies of the prior art by eliminating the clotting that may be caused in the access port or arteriovenous fistula, resulting in a more durable vascular access system, a decreased number of surgical procedures over the lifetime of the patient, and decreased cost of medical care for hemodialysis patients.

### SUMMARY OF THE INVENTION

The subject invention is directed to a new and improved apparatus for vascular access to facilitate hemodialysis. The apparatus includes a vascular access port defining an elongated tubular body of predetermined length with a central lumen having opposed proximal and distal end portions. The distal end portion of the body is adapted and configured for introduction into a blood vessel. The apparatus further includes an elongated cylindrical plug body dimensioned and configured for insertion into the central lumen of the vascular access port. In accordance with the subject invention, the plug body has a length that is substantially equal to the length of the vascular access port so as to prevent blood flow into the lumen of the access port when the plug is engaged therein.

Preferably, a handle portion and locking mechanism are operatively associated with the proximal end of the elongated cylindrical plug body. The handle portion can be used to facilitate installation and removal of the plug body. The locking mechanism provides means for coupling and sealingly engaging the plug body to the vascular access port. In one embodiment of the invention, the plug body, handle portion and locking mechanism are formed monolithically. In another embodiment of the invention, the plug body, handle portion and locking mechanism are integral with one another.

It is envisioned that the locking mechanism is adjacent to and attached to the elongated cylindrical plug body for coupling the plug body to the vascular access port. The locking means may comprise helical threads which can extend radially beyond the outer diameter of the elongated cylindrical plug body. Alternatively, the locking means may include a luer lock fitting or at least one protuberance on the proximal end of the plug body that is adapted and configured for insertion into a corresponding recess disposed at the proximal end of the vascular access port.

In accordance with a preferred embodiment of the subject invention, the elongated cylindrical plug body has an outer diameter that is substantially equal to the inside diameter of the lumen of the vascular access port, and the handle portion extends radially outward from the outer diameter of the plug body. It is also envisioned that the elongated cylindrical plug body has a central core to increase the flexibility of the plug body, and a fitting is provided at the proximal end of the elongated cylindrical plug body for facilitating locking. The proximal end of the elongated cylindrical plug body may be fluted to provide a friction fit, or knurled to provide an interference fit.

The subject invention is also directed to a vascular access system that includes, inter alia, a first vascular access port for providing ingress of fluid into a blood vessel, a second vascular access port for providing egress of fluid from a blood vessel, a first elongated cylindrical plug body dimensioned and configured for insertion into the central lumen of the first vascular access port, and a second elongated cylindrical plug body dimensioned and configured for insertion into the central lumen of the second vascular access port.

The subject invention is also directed to a kit which includes a vascular access port defining an elongated tubular body of predetermined length with a central lumen having opposed proximal and distal end portions, the distal end portion adapted and configured for introduction into a blood vessel. The kit also includes an elongated cylindrical plug body dimensioned and configured for insertion into the central lumen of the vascular access port. The kit still further includes an enclosure adapted and configured to support a plurality of vascular access ports and a plurality of elongated cylindrical plug bodies. Preferably, the kit further includes a plurality of stabilizer pads each having a central aperture adapted and configured to allow insertion of a vascular access port which is adhesively attached to a patient's skin.

Those skilled in the art will readily appreciate that the subject invention decreases patient suffering, extends the life of the access ports and associated grafts, decreases the number of surgical procedures over the lifetime of the patient, decreases the cost of medical care for hemodialysis, and decreases damage to the entire venous system of the patient with the decreased need for access port relocations.

These and other unique features of the subject invention will become more readily apparent to those having ordinary skill in the art from the following description of the drawings taken in conjunction with the detailed description of the preferred embodiments.

### BRIEF DESCRIPTION OF THE DRAWINGS

So that those having ordinary skill in the art to which the subject invention appertains will more readily understand how to construct and use the subject invention, reference may be had to the drawings wherein:

FIG. 1 is an illustration of a prior art vascular access system for hemodialysis having first and second access ports that include end caps;

FIG. 2 is a cross-sectioned view of a portion of the prior art vascular access system of FIG. 1 in which a capped access port is disposed within a blood vessel;

FIG. 3 is a perspective view of an apparatus for facilitating vascular access configured in accordance with a preferred embodiment of the subject invention with parts separated for ease of illustration, the apparatus including an elongated cylindrical plug body and a vascular access port;

FIG. 4 is a cross-sectioned view of an apparatus for facilitating vascular access as illustrated in FIG. 3, wherein the length of the elongated cylindrical plug body is substantially equal to the length of the vascular access port;

FIG. 5 is a perspective view of an apparatus for facilitating vascular access configured in accordance with a preferred embodiment of the subject invention with parts separated for ease of illustration, wherein a handle portion and locking mechanism are associated with a proximal end of the elongated cylindrical plug body;

FIG. 6 is a cross-sectioned view of an apparatus for facilitating vascular access as illustrated in FIG. 5, wherein the locking mechanism associated with the proximal end of the elongated cylindrical plug body includes helical threads which extend radially beyond the outer diameter of the plug body;

FIG. 7 is a perspective view of an apparatus for facilitating vascular access configured in accordance with a preferred embodiment of the subject invention with parts separated for ease of illustration, wherein the locking mechanism includes a protuberance on the proximal external end of the plug body adapted and configured for insertion into a corresponding recess disposed at the proximal end of the vascular access port;

FIG. 8 is a cross-sectioned view of an apparatus for facilitating vascular access as illustrated in FIG. 7, wherein the elongated cylindrical plug body has a central core;

FIG. 9 is a perspective view of an apparatus for facilitating vascular access configured in accordance with a preferred embodiment of the subject invention with parts separated for ease of illustration, wherein the proximal end of the elongated cylindrical plug body is textured to provide a friction fit;

FIG. 10 is a cross-sectioned view of an apparatus for facilitating vascular access as illustrated in FIG. 9, wherein the elongated cylindrical plug body has a solid core and is engaged within the vascular access port by linear insertion as indicated by the directional arrows;

FIGS. 11 through 14 illustrate the operative steps for utilizing an apparatus for facilitating vascular access configured in accordance with a preferred embodiment of the subject invention, wherein:

FIG. 11 is a cross-sectioned view of an apparatus as illustrated in FIG. 4, wherein the elongated cylindrical plug body is engaged in the vascular access port so as to prevent the flow of blood into the lumen of the access port;

FIG. 12 is a cross-sectioned view of the apparatus as illustrated in FIG. 4, wherein the elongated cylindrical plug body is being removed from within the vascular access port, with only a portion remaining partially engaged;

FIG. 13 is a cross-sectioned view of an apparatus for facilitating vascular access as illustrated in FIG. 4 which is connected to hemodialysis machine and is providing access to the patient's blood flow;

FIG. 14 is a cross-sectioned view of an apparatus for facilitating vascular access as illustrated in FIG. 4, wherein the elongated cylindrical plug body is being inserted into the vascular access port and preventing blood from remaining in the lumen of the access port; and

FIG. 15 is a perspective view of a kit constructed in accordance with the subject invention which includes an enclosure containing vascular access ports, elongated cylindrical plug bodies, and stabilizer pads.

These and other features of the subject invention will become more readily apparent to those having ordinary skill in the art from the following detailed description of the preferred embodiments.

### DETAILED DESCRIPTION OF PREFERRED EMBODIMENTS

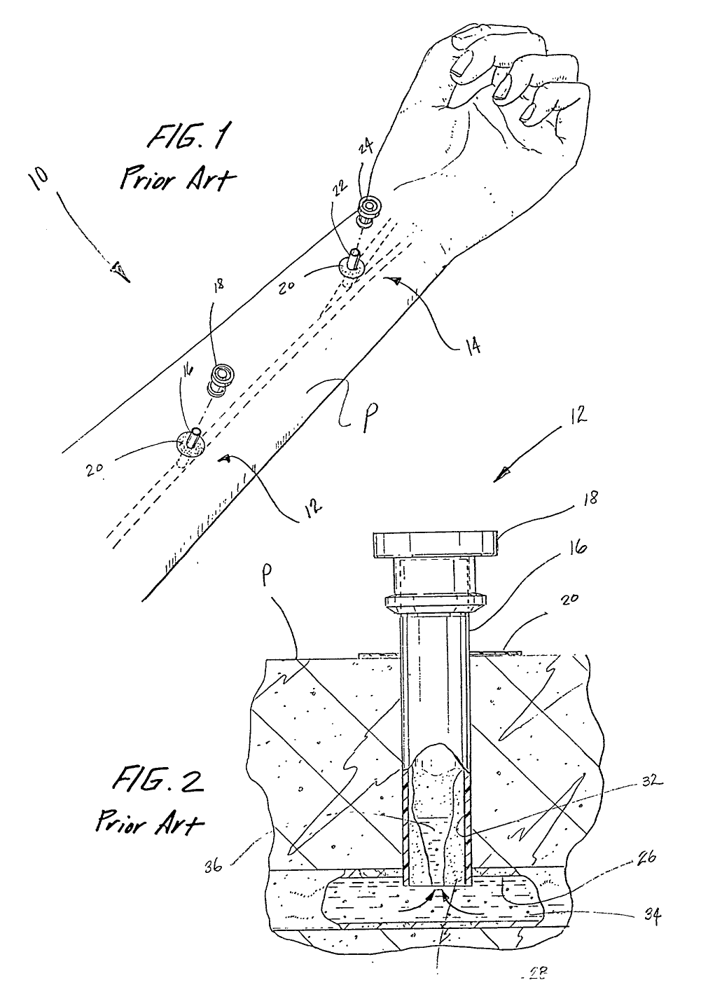

Referring now to the drawings wherein like reference numerals identify similar structural elements of the subject invention, there is illustrated in FIG. 1 a prior art vascular access system for hemodialysis designated generally by reference numeral 10. Vascular access system 10 includes inlet zone 12 and outlet zone 14. Percutaneous inlet port 16 and percutaneous outlet port 22 are implanted in the arm of a patient P at zones 12 and 14 respectively. When the dialysis process is suspended and access to the blood flow of patient P is not required, end caps 18 and 24 are used to cover the percutaneous inlet port 16 and percutaneous outlet port 22 ports respectively. Circular stabilizer pads 20 are also included in the prior art vascular access system 10 and are installed over the percutaneous inlet port 16 and percutaneous outlet 22 port. Stabilizer pads 20 are adhesively attached to the skin of patient P and prevent movement of the percutaneous ports, damage to the surrounding skin tissue and reduce the chance of infection.

Referring to FIG. 2, wherein a portion of the prior art vascular access system 10 of FIG. 1 is illustrated in cross-sectioned view. Percutaneous inlet port 16 is shown disposed within the arm of patient P. A stabilizer pad 20 is installed over the inlet port 16 to prevent its movement. As shown, access to the blood 34 of patient P is not required. Therefore, end cap 18 is affixed over the proximal end of inlet port 16. End cap 18 prevents blood 34 from flowing through the lumen 32 of inlet port 16, but allows stagnant blood 36 to remain in the lumen 32 of the vascular access port 16. Stagnant blood 36 may cause residual debris 28 to accumulate in the distal end of the lumen 32 reducing the available flow rate for dialysis.

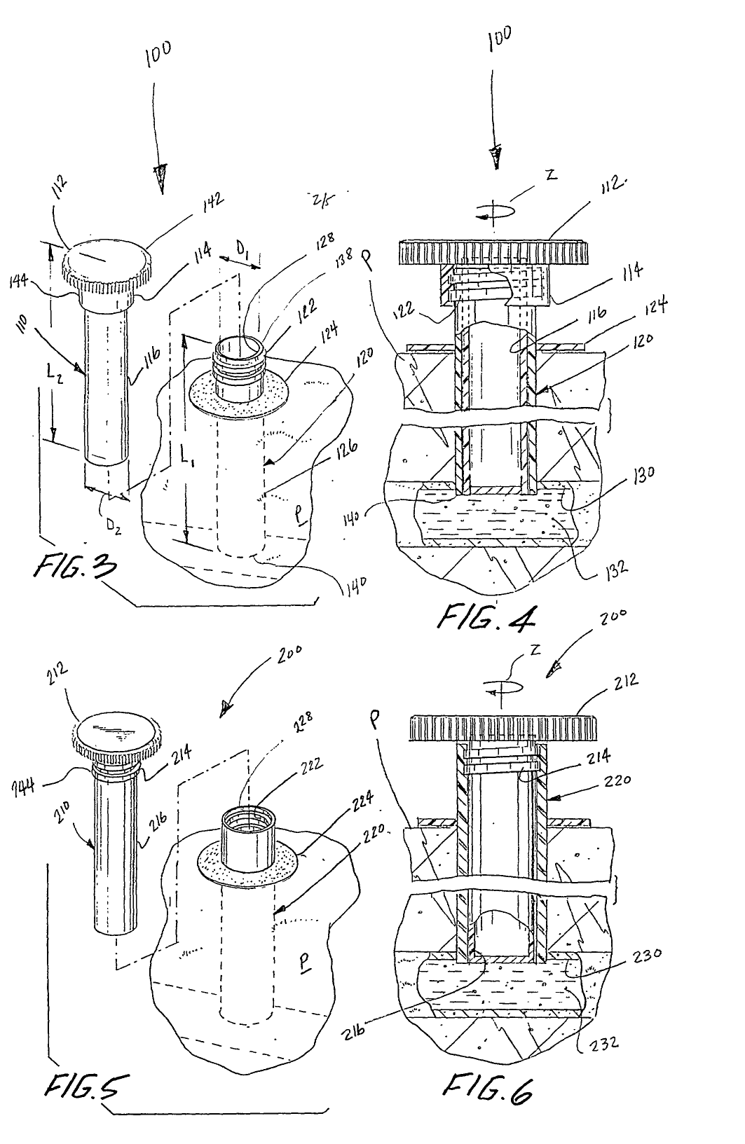

Referring now to FIGS. 3 and 4, there is illustrated an apparatus for facilitating vascular access constructed in accordance with a preferred embodiment of the subject invention and designated generally by reference numeral 100. Apparatus 100 includes a vascular access port 120 defining an elongated tubular body 126 of predetermined length L₁ with a central lumen 128 having opposed proximal and distal end portions 138 and 140 respectively. Apparatus 100 further includes an elongated cylindrical plug body 110 dimensioned and configured for insertion into the central lumen 128 of the vascular access port 120. The plug body 110 has a length L₂ that is substantially equal to the length L₁ of the vascular access port 120. In a preferred embodiment, the length L₁ of the vascular access port 120 may be between 10 and 20 centimeters.

The distal end portion 140 of vascular access port 120 is adapted and configured for introduction into blood vessel 130. The lumen 128 of the vascular access port 120 provides a conduit for the flow of blood 132 from the blood vessel 130 of patient P to medical equipment such as a hemodialysis machine (not shown).

The elongated cylindrical plug body 110 has an outer diameter D₂ that is substantially equal to an inside diameter D₁ of the lumen 128 of the vascular access port 120. Therefore, when the plug body 110 is inserted into the lumen 128 of the access port 120, during periods in which the dialysis process is suspended, the flow of blood 132 into the access port 120 is prevented. Additionally, since the length L₂ of the plug body 110 is substantially equal to the length L₁ of the access port 120, stagnant blood 143 will not remain in the lumen 128 nor will residual debris accumulate therein.

With continuing reference to FIGS. 3 and 4, handle portion 112 is associated with the proximal end 142 of the elongated cylindrical plug body 110 to facilitate installation and removal of the plug body 110. The handle portion 112 extends radially outward from plug body 110. Also associated with the proximal end 142 of the elongated cylindrical plug body 110 is locking mechanism 114 which comprises a tubular body 144 having threads formed internally. The tubular body 144 and internal threads being adapted an configured for receiving threads 122 located on the proximal end 138 of the vascular access port 120 and sealingly engaging therewith. The locking mechanism is engaged by grasping handle portion 112 and rotating the plug body 110 clockwise, as shown by directional arrow Z.

Although the plug body 110 would normally be disposed within the arm of the patient P, those skilled in the art will readily appreciate that the port can be use to obtain vascular access in other areas of the body, such as the chest or legs.

Referring now to FIGS. 5 and 6, there is illustrated an apparatus for facilitating vascular access constructed in accordance with another embodiment of the subject invention and designated generally by reference numeral 200. Apparatus 200 includes a vascular access port 220 and an elongated cylindrical plug body 210 and is substantially similar in structure and function to apparatus 100 shown in FIGS. 3 and 4. It differs however, in that the locking mechanism 214 includes helical external threads 244 associated with the outer diameter D₂ of the plug body 210. Additionally, in this embodiment, internal threads 222 are associated with the lumen 228 of the access port 220 and are adapted and configured for receiving external threads 244 of plug body 210. Like apparatus 100, the locking mechanism is engaged by grasping handle portion 212 and rotating the plug body 210 clockwise, as shown by directional arrow Z in FIG. 6.

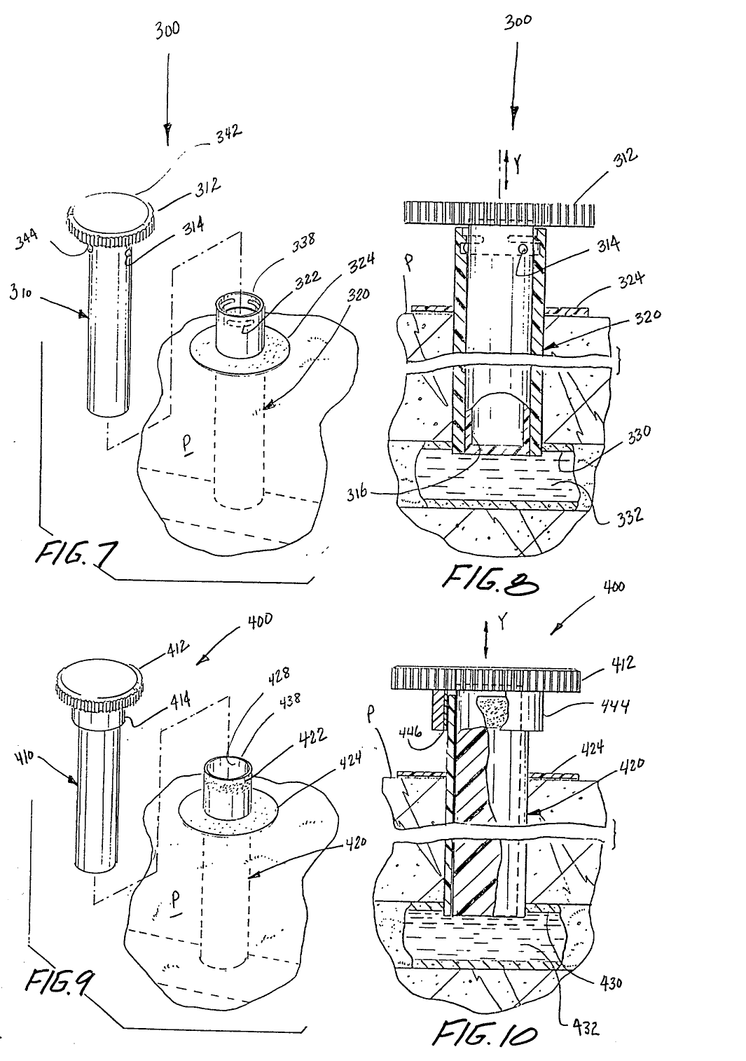

Referring now to FIGS. 7 and 8, there is illustrated an apparatus for facilitating vascular access constructed in accordance with still another embodiment of the subject invention and designated generally by reference numeral 300. Apparatus 300 includes a vascular access port 320 and an elongated cylindrical plug body 310 and is substantially similar in structure and function to the apparatus shown in FIGS. 3 and 4. This apparatus differs however, in that locking mechanism 314 includes at least one protuberance 344 disposed on the proximal end 342 of the plug body 310 and adapted and configured for insertion into a corresponding recess 322 disposed at the proximal end 338 of the vascular access port 320. The locking mechanism is engaged by grasping handle portion 112 and linearly inserting plug body 310 into vascular access port 320, as shown by directional arrow y, until protuberance 344 is positioned within corresponding recess 322.

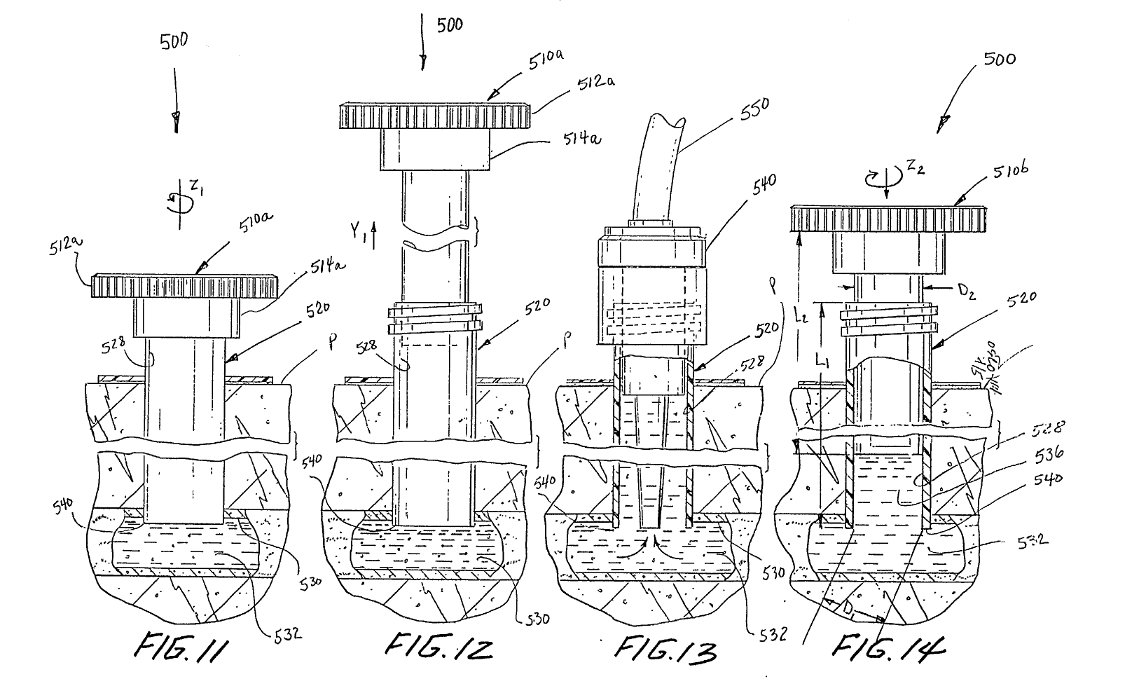

Referring to FIGS. 9 and 10, there is illustrated an apparatus for facilitating vascular access constructed in accordance with yet another embodiment of the subject invention and designated generally by reference numeral 400. The apparatus for facilitating vascular access 400 includes a vascular access port 420 and an elongated cylindrical plug body 410 and is substantially similar in structure and function to the apparatus shown in FIGS. 3 and 4. It differs however, in that the locking mechanism 414 includes a tubular structure 444 having a textured surface 446 disposed on the internally. The locking mechanism 414 is adapted and configured for engagement with a corresponding textured surface 422 disposed on the proximal end 438 of vascular access port 420. It is engaged by inserting the plug body 410 into the lumen 428 of the vascular access port 420, as shown by directional arrow Y, until handle portion 412 contacts the proximal end 438 of the vascular access port 420.

Referring to FIGS. 11 through 14, there is illustrated the operative steps for using an apparatus constructed in accordance with the subject invention such as the embodiment shown in FIGS. 3 and 4. The apparatus shown in FIGS. 11 through 14 is designated generally by reference numeral 500. Vascular access is accomplished by first implanting the vascular access port 520 in the arm of patient P or other suitable vascular access site, by using a percutaneous technique.

A technique for implanting access port 520 may include several steps that are not detailed in the drawings but are well known to those skilled in the art. First, a needle is inserted into the blood vessel at the desired location and its position is verified by observing fluid return or by a similar method. While the needle is held firmly in place a guidewire is inserted through the needle cannula to the desired depth. The guidewire is then held in place and the needle is withdrawn. Pressure is applied on the puncture site in order to minimized blood loss. Next, an introducer/sheath assembly is threaded over the guide wire. The introducer/sheath assembly is grasped close to the skin surface and advanced through the tissue, to the desired position. Then, the introducer and guidewire are removed, leaving the sheath installed. The vascular access port is then introduced into the sheath and advanced to the desired position. The sheath is next peeled apart and removed, leaving the access port disposed within the blood vessel of patient P.

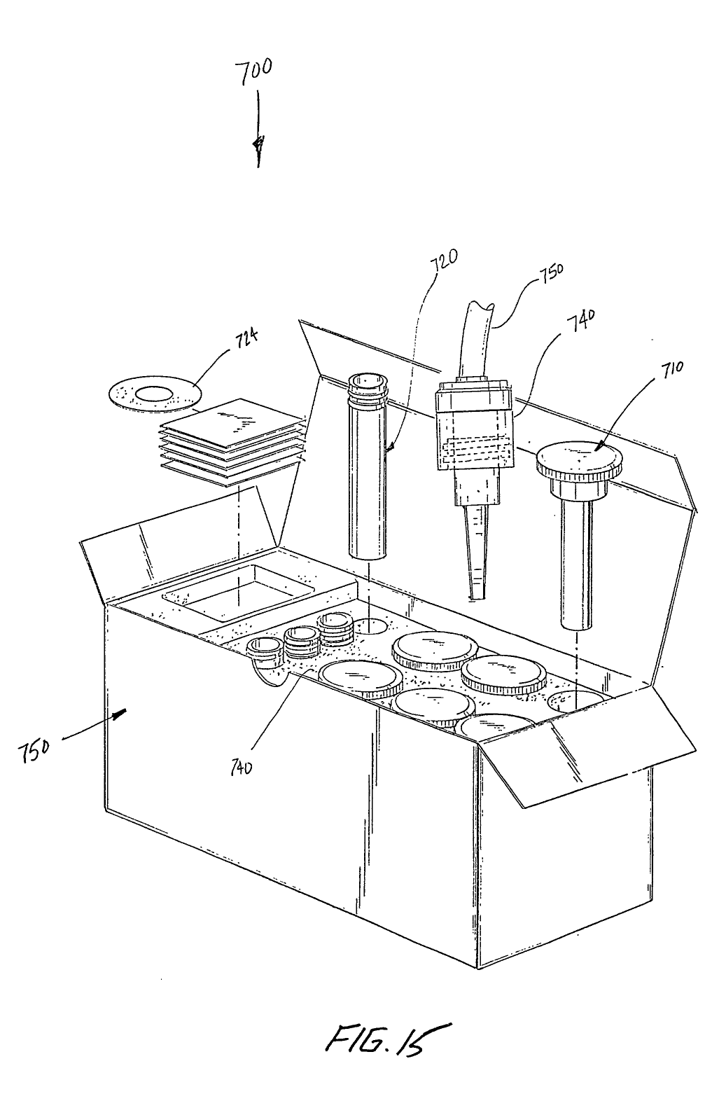

Referring to FIG. 11, in use elongated cylindrical plug body 510a is engaged in lumen 528 of access port 520 and the flow of blood 532 through the access port 520 is suspended. Access to the flow of blood is achieved by first disengaging locking mechanism 514a. This can be accomplished by grasping handle portion 512a and rotating the plug body 510a counter-clockwise, as shown by directional arrow Z₁. Then, plug body 510a is removed from the access port 520 and discarded as indicated by directional arrow Y₁. As shown in FIG. 12, the elongated cylindrical plug body 510a partially is withdrawn from the vascular access port 520. Once the plug body 510a is completely removed from access port 520, connector 540 and associated conduit 550 is engaged with the proximal end of access port 520, providing a continuous flowpath from the blood vessel 530 to the hemodialysis equipment (not shown). A vascular access port 520 having connector 540 and conduit 550 associated therewith is shown in FIG. 13.

Once the dialysis procedure is complete, connector 540 and associated conduit 550 are disengaged from the proximal end of the access port 520. Then, a new plug body 510b is inserted into lumen 528 and locking mechanism 514 is engaged by rotating the plug body 510b in a clockwise manner as indicated by directional arrow Z₂. The new plug body 510b is partially inserted into access port 520 as shown in FIG. 14. Since the plug body 510b has a length L₂ and diameter D₂ that is substantially equal to length L₁ and diameter D₂ of the vascular access port 520, blood 536 is forced from the lumen 528 of the access port 520 preventing debris from accumulating and reducing the available dialysis flow rate.

Referring to FIG. 15, there is illustrated a kit constructed in accordance with a preferred embodiment of the subject invention and designated generally by reference numeral 700. The kit 700 includes enclosure 750 which contains a plurality of vascular access ports 720a through 720d and elongated cylindrical plug bodies 710a through 710f. Preferably, the enclosure 750 contains foam core material 740 having recesses adapted and configured to support the access ports 720a through 720d and plug bodies 710a through 710f. Ideally, the kit 700 further includes stabilizer pads 724 and multiple connectors 740 with associated conduit 750. Stabilizer pads 724 adhesively attach to the patient's skin and prevent movement of the access port, damage to the surrounding skin tissue and reduce the chance of infection. It is envisioned that alternate packing materials such as blow molded or injection molded plastic may be used.

Although the disclosed apparatus has been described with respect to preferred embodiments, it is apparent that modifications and changes can be made thereto without departing from the spirit and scope of the invention as defined by the appended claims.

What is claimed is:

1. An apparatus for facilitating vascular access comprising:
a) a vascular access port defining an elongated tubular body of predetermined length with a central lumen having opposed proximal and distal end portions, the distal end portion adapted and configured for introduction into a blood 5 vessel; and
b) an elongated cylindrical plug body dimensioned and configured for insertion into the central lumen of the vascular access port, the plug body having a length that is substantially equal to the length of the vascular access port so as to prevent blood flow into the lumen of the access port when the plug is engaged therein.

2. An apparatus as recited in claim 1, wherein a handle portion is associated with a proximal end of the elongated cylindrical plug body to facilitate installation and removal of the plug body.

3. An apparatus as recited in claim 2, wherein a locking mechanism is associated with the proximal end of the elongated cylindrical plug body for coupling the plug body to the vascular access port.

4. An apparatus as recited in claim 3, wherein the plug body, handle portion and locking mechanism are formed monolithically.

5. An apparatus as recited in claim 3, wherein the plug body, handle portion and locking mechanism are integral with one another.

6. An apparatus as recited in claim 1, wherein a locking mechanism is adjacent to and attached to the elongated cylindrical plug body for coupling the plug body to the vascular access port.

7. An apparatus as recited in claim 6, wherein the plug body and locking mechanism are formed monolithically with one another.

8. An apparatus as recited in claim 6, wherein the plug body, and locking mechanism are integral with one another.

9. An apparatus as recited in claim 1, wherein the elongated cylindrical plug body has an outer diameter that is substantially equal to an inside diameter of the lumen of the vascular access port.

10. An apparatus as recited in claim 2, wherein the handle portion extends radially outward from an outer diameter of the plug body.

11. An apparatus as recited in claim 6, wherein the locking mechanism comprises means for sealingly engaging the vascular access port.

12. An apparatus as recited in claim 11, wherein the locking means comprises helical threads.

13. An apparatus as recited in claim 12, wherein the helical threads extend radially beyond the outer diameter of the elongated cylindrical plug body.

14. An apparatus as recited in claim 11, wherein the locking means includes a luer lock fitting.

15. An apparatus as recited in claim 11, wherein the locking mechanism comprises at least one protuberance on the proximal end of the plug body adapted and configured for insertion into a corresponding recess disposed at the proximal end of the vascular access port.

16. An apparatus as recited in claim 1, wherein the elongated cylindrical plug body has a central core to increase flexibility of the plug body.

17. An apparatus as recited in claim 1, wherein a fitting is provided at the proximal end of the elongated cylindrical plug body for facilitating locking.

18. An apparatus as recited in claim 1, wherein the proximal end of the elongated cylindrical plug body is fluted to provide a friction fit.

19. An apparatus as recited in claim 1, wherein the proximal end of the elongated cylindrical plug body is knurled to provide an interference fit.

20. A vascular access system comprising:
a) a first vascular access port for providing ingress of fluid into a blood vessel, defining an elongated tubular body of predetermined length with a central lumen having opposed proximal and distal end portions, the distal end portion adapted and configured for introduction into a blood vessel;
b) a second vascular access port for providing egress of fluid from a blood vessel, defining an elongated tubular body of predetermined length with a central lumen having opposed proximal and distal end portions, the distal end portion adapted and configured for introduction into a blood vessel;
c) a first elongated cylindrical plug body dimensioned and configured for insertion into the central lumen of the first vascular access port, the first plug body having a length substantially equal to the length of the first vascular access port, so as to prevent blood flow into the lumen of the first access port when the plug is engaged therein; and
d) a second elongated cylindrical plug body dimensioned and configured for insertion into the central lumen of the second vascular access port, the second plug body having a length substantially equal to the length of the second vascular access port, so as to prevent blood flow into the lumen of the second access port when the plug is engaged therein.

21. A kit comprising:
a) a vascular access port defining an elongated tubular body of predetermined length with a central lumen having opposed proximal and distal end portions, the distal end portion adapted and configured for introduction into a blood vessel;
b) an elongated cylindrical plug body dimensioned and configured for insertion into the central lumen of the vascular access port, the plug body having a length substantially equal to the length of the vascular access port, so as to prevent blood flow into the lumen of the access port when the plug is engaged therein; and
c) an enclosure, adapted and configured to support a vascular access port and an elongated cylindrical plug body.

22. A kit as recited in claim 21, further comprising a stabilizer pad having a central hole adapted and configured to allow insertion over a vascular access port and adhesively attached to a patient's skin.

23. A kit as recited in claim 21, wherein the enclosure is adapted and configured to support a stabilizer pad.

24. A kit as recited in claim 21, wherein the enclosure is adapted and configured to support a connector assembly and conduit.
